(19) United States
(12) Patent Application Publication
(10) Pub. No.: US 2013/0237709 A1
(43) Pub. Date: Sep. 12, 2013

(54) INTERMEDIATES USEFUL FOR THE SYNTHESIS OF FEXOFENADINE, PROCESSES FOR THEIR PREPARATION AND FOR THE PREPARATION OF FEXOFENADINE

(71) Applicant: CHEMELECTIVA S.R.L., Novara (IT)

(72) Inventors: Graziano CASTALDI, Briona (NO) (IT); Marco BARATELLA, Cerano (NO) (IT); Mauro GABOARDI, Novara (IT)

(73) Assignee: CHEMELECTIVA S.r.l., Novara (IT)

(21) Appl. No.: 13/772,832

(22) Filed: Feb. 21, 2013

(30) Foreign Application Priority Data
Mar. 12, 2012 (IT) MI2012A000329
Apr. 21, 2012 (IT) MI2012A000589

(51) Int. Cl. C07D 307/20; C07D 211/22; C07C 67/313

(52) U.S. Cl. CPC C07D 307/20; C07C 67/313; C07D 211/22
USPC 546/239; 549/475; 560/51

(57) ABSTRACT

Intermediates useful for the synthesis of fexofenadine, processes for their preparation and processes for the synthesis of fexofenadine are described.

### CROSS-REFERENCE TO RELATED APPLICATIONS

This Application claims the benefit of priority from Italian Patent Application Nos. MI2012A000329, filed Mar. 2, 2012 and MI12012A000589, filed Apr. 12, 2012, the contents of each of which are incorporated herein by reference.

### FIELD OF THE INVENTION

The present invention relates to new intermediates useful for the synthesis of fexofenadine, processes for their preparation and their conversion to fexofenadine.

### BACKGROUND OF THE INVENTION

Fexofenadine is a so-called “second generation” oral antihistamine agent, devoid of side effects, used for treating the symptoms of allergic reactions, symptoms related to the release of histamine. Histamine is one of the chemical mediators of inflammation, deriving from the decarboxylation of histidine by histidine decarboxylase. Antihistamine agents inhibit the activity of H1 histamine receptors, mainly present in the skin and in the bronchi, by blocking histamine release. The contact of an allergen (a substance which can cause allergic symptoms) with H1 histamine receptors stimulates the release of histamine, causing capillary dilatation, capillary permeability and vasodilatation, then causing local erythemas, local edemas (swellings) and rashes. Furthermore, following histamine release, itching and, in predisposed subjects, severe bronchoconstrictions, gastroenteric mobility and an increase of salivary and bronchial gland secretion may occur.

Fexofenadine is the carboxylic acid corresponding to terfenadine, of which is the main metabolite. However, fexofenadine does not show the cardiotoxic effects of terfenadine and, thanks to the fact that it does not cross the blood-brain barrier, it does not cause to the patient those side effects typical of the so-called “first generation” antihistamine agents, such as sleepiness and tiredness. Fexofenadine is the compound of formula (I)

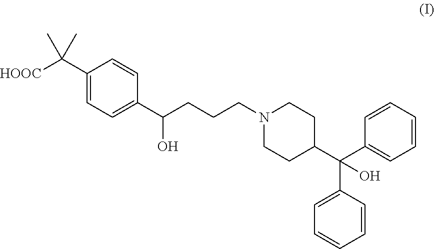 [SMILES: CC(C)(C(=O)O)c1ccc(C(O)CCCN2CCC(C(O)(c3ccccc3)c3ccccc3)CC2)cc1]

chemically known as 2-[4-[1-hydroxy-4-[4-(hydroxy-diphenyl-methyl)-1-piperidyl]butyl]phenyl]-2-methyl-propanoic acid, described in U.S. Pat. No. 4,254,129 and sold under the trademark Allegro®.

WO 02/10115 (Texcontor Etablissement) discloses a process for the synthesis of fexofenadine comprising the reaction of the compound of formula (II)

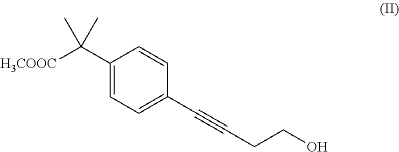

with a copper or silver compound, in particular copper(II)oxide in the presence of palladium, or of a compound containing palladium, to give the intermediate of

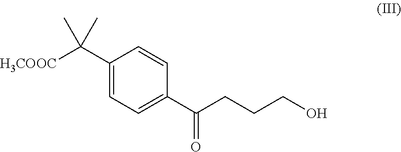

CN 102079708 (Zhejiang Giming Pharmaceutical Co ltd) discloses a process for the synthesis of fexofenadine comprising the preparation of the compound of formula (IV)

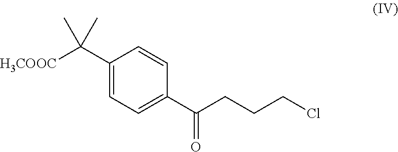

by reacting the intermediate of formula (II) with a mercury(II)compound, in particular mercury(II)oxide.

Both processes are characterized by the use of expensive and difficult to handle reagents, because of their high toxicity by inhalation, contact with the skin and ingestion, and of their high damaging action to the environment.

### SUMMARY OF THE INVENTION

We have now found new compounds which are useful intermediates for the synthesis of fexofenadine and which do not require the use of toxic reagents such as mercury salts or expensive reagents such as palladium salts and which allow to use easy handling reaction conditions with low environmental impact.

Therefore, an object of the present invention are the compounds of formula (V)

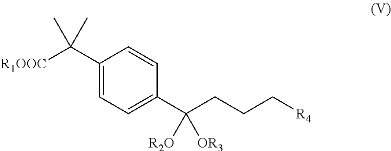

wherein
R₁, R₂ and R₃, the same or different, are linear or branched C₁-C₂₀ alkyl groups;
or R₂ and R₃ linked together are a five or six membered ring of formula:

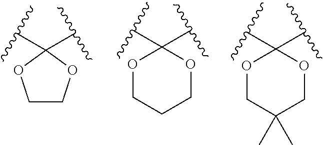

R₄ is a halogen atom or a hydroxy group; or
the groups OR₂ and R₄ linked together are a five membered cycle of formula.

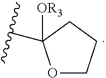

A preferred object of the present invention are the compounds of formula (Va)

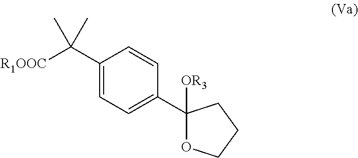

wherein
R₁ and R₃, the same or different, are linear or branched C₁-C₂₀ alkyl groups.

In particular, a still more preferred object of the present invention is a compound of formula (Vb)

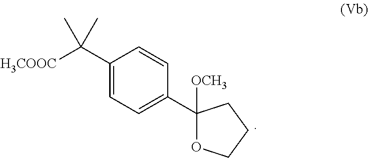

The compounds of formula (V), (Va) and (Vb) are new and are useful intermediates for the synthesis of fexofenadine.

A further object of the present invention is a process for the synthesis of the compounds of formula (V), preferably (Va), comprising the dissolution of the intermediates of formula (IIa)

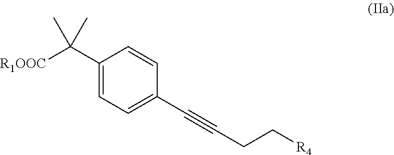

wherein R₁ is a linear or branched C₁-C₂₀ alkyl group and R₄ is a halogen or a hydroxy group, in an alcoholic solvent and the subsequent reaction with a Brønsted acid, or a Lewis acid, or mixtures thereof, at a temperature from 25° C. to the reflux temperature of the solvent for a time sufficient to form the desired compounds which are recovered using known techniques,

The alcoholic solvent is a linear or branched C₁-C₄ alcohol, preferably selected from among methanol, ethanol, isopropanol and butanol. Methanol is preferably used.

The Brønsted acid is preferably selected from among hydrochloric acid, hydrobromic acid, sulfuric acid.

The Lewis acid is preferably selected from among zinc salts, preferably bromide, chloride, oxide, acetate, sulfate.

Preferably, a mixture of a Brønsted acid and a Lewis acid, still more preferably a mixture of hydrochloric acid and zinc chloride, is used.

The amount of hydrochloric acid is from about 1 to about 5 equivalents and the amount of zinc chloride is from about 1 and about 5 equivalents. About 2 equivalents of hydrochloric acid and about 3 equivalents of zinc chloride are preferably used.

The compounds of formula (V) can also be prepared starting from an intermediate of formula (VI)

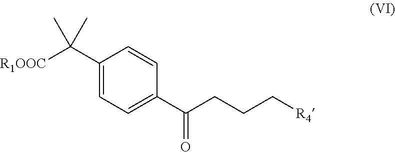

wherein R₁ is a linear or branched C₁-C₂₀ alkyl group and R₄′ is a halogen atom, preferably chlorine or bromine.

A further object of the present invention is a process for the preparation of the compounds of formula (VI) starting from a compound of formula (IIa) according to the following scheme:

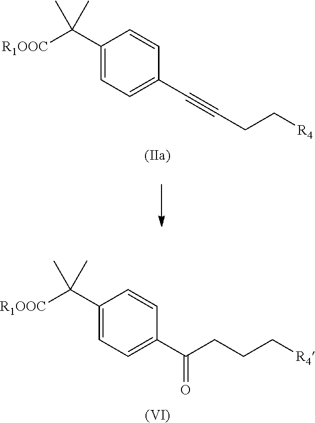

wherein
R₁ is a linear or branched C₁-C₂₀ alkyl group;
R₄ is a halogen atom or a hydroxy group; and
R₄ is a halogen atom;
   by reaction with a hydrogen halide solution in a suitable solvent at a temperature from 30° C. to 50° C.

Suitable hydrogen halide solutions are a solution of hydrobromic acid in acetic acid and an aqueous hydrochloric acid solution.

A preferred embodiment of the present invention is the synthesis of the compound of formula (Vb)

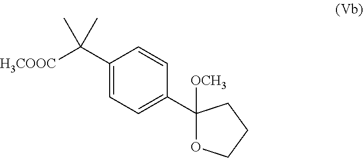

comprising the dissolution of the intermediate of formula (H)

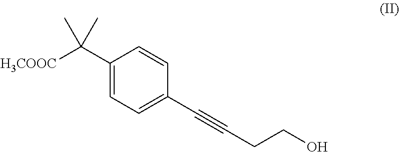

in methanol and the subsequent addition of zinc chloride and hydrochloric acid, at the reflux temperature of the solvent.

A further preferred embodiment of the present invention is the synthesis of a compound of formula (VIa)

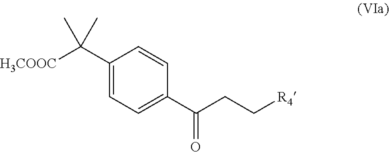

wherein
R₄′ is chlorine or bromine, according to the following scheme:

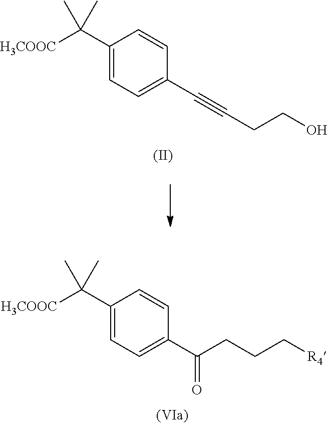

by reaction with a hydrobromic acid or hydrochloric acid solution in a suitable solvent at a temperature from 30° C. to 50° C.

A further object of the present invention is a process for the synthesis of fexofenadine, starting from intermediates of formula (V), comprising:
reacting an intermediate of formula (V) with aqueous hydrobromic acid or acetic acid in the presence of a solvent, preferably toluene, to give an intermediate of formula (VI)

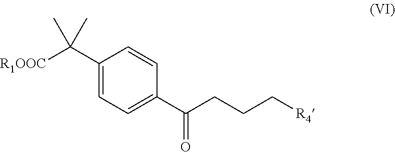

wherein R₁ is a linear or branched C₁-C₂₀ alkyl group and R₄′ is a halogen atom; and
transforming the intermediate of formula (VI) into fexofenadine.

The intermediates of formula (VI) and their transformation into fexofenadine are described, for example, in U.S. Pat. No. 6,147,216, the disclosure of is which is incorporated herein by reference.

Fexofenadine is preferably prepared starting from intermediates of formula (Va).

A further object of the present invention is a process for the synthesis of fexofenadine starting from intermediates of formula (VI), comprising:
a) transforming an intermediate of formula (VI) into a compound of formula (Vc)

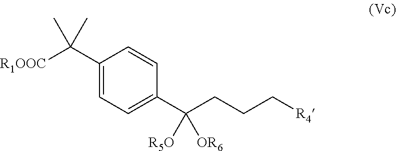

wherein
R₁, R₅ and R₆, the same or different, are linear or branched C₁-C₂₀ alkyl groups and R₄′ is a halogen atom; by reaction with a trialkylorthoformate in an alcoholic solvent in the presence of an acid catalyst at a temperature from 25° C. to the reflux temperature of the solvent:
b) reacting the resultant compound of formula (Vc) with azacyclonol in a suitable solvent in the presence of a base at a temperature from 20° C. to the reflux temperature of the solvent, followed by the optional saponification and by the optional subsequent treatment with acetic acid to give a compound of formula (VII)

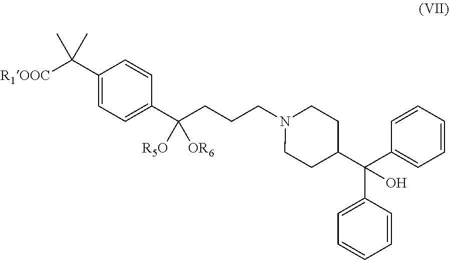

wherein R₁′ is a hydrogen atom or a linear or branched C₁-C₂₀ alkyl group; and
R₅ and R₆, the same or different, are linear or branched C₁-C₂₀ alkyl groups;
c) reacting the resultant compound (VII) with an aqueous acid in the presence of a suitable solvent at a temperature from 20° C. to 40° C. to give the compound of formula (VIII)

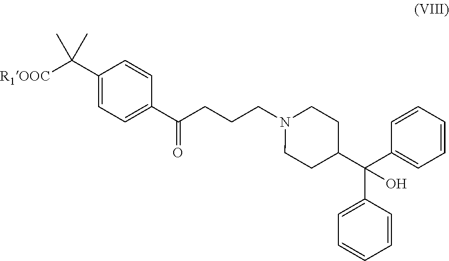

wherein R₁ is a hydrogen atom or a linear or branched C₁-C₂₀ alkyl group; and
d) transforming the resultant compound (VIII) into fexofenadine.

The starting intermediate (VI) can be prepared from a compound of formula (Va) by reaction with aqueous hydrobromic acid or acetic acid in a suitable solvent, preferably toluene, or from a compound of formula (IIa) by reaction with a hydrohalide acid solution in a suitable solvent as above described.

The transformation of compounds (VIII) into fexofenadine is carried out according to the method described in WO 2011/158262, the contents of which are incorporated herein by reference, in the name of the Applicant.

When the saponification reaction is not carried out in step b) of the process object of the present invention, said reaction can be carried out in one of the subsequent steps c) and d). The saponification reaction is carried out according to known techniques, preferably in the presence of sodium or potassium hydroxide in methanol.

The trialkylorthoformate in step a) is selected from among trimethylorthoformate, triethylorthoformate or triisopropylorthoformate. Trimethylorthoformate is preferably used.

The alcoholic solvent is a linear or branched C₁-C₄ alcohol, preferably selected from among methanol, ethanol, isopropanol, butanol. Methanol is preferably used.

The acid catalyst in step a) is preferably selected from sulfuric acid, camphorsulfonic acid and methansulfonic acid. Camphorsulfonic acid is preferably used.

The solvent in step b) is preferably selected from among toluene, acetonitrile, tetrahydrofuran. Toluene is preferably used.

The base is preferably selected from among sodium bicarbonate, potassium bicarbonate, triethylamine. Sodium bicarbonate is preferably used.

The aqueous acid in step c) is preferably selected from among sulfuric acid, hydrochloric acid. Sulfuric acid is preferably used.

The solvent in step c) is preferably selected from among methanol, ethanol, isopropanol. Methanol is preferably used.

A preferred embodiment of the process object of the present invention is a process for the synthesis of fexofenadine starting from intermediates of formula (VIa),

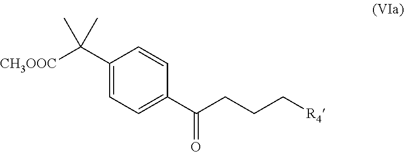

wherein R₄′ is a halogen atom;
comprising:
a′) transforming an intermediate of formula (VIa) into a compound of formula (Vd)

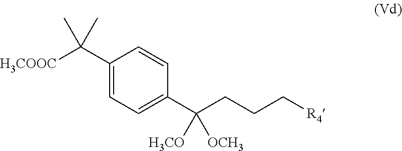

wherein R₄′ is a halogen atom;
by reaction with a trialkylorthoformate in an alcoholic solvent in the presence of an acid catalyst at a temperature from 25° C. to the reflux temperature of the solvent;
b′) reacting the resultant compound of formula (Vd) with azacyclonol in a suitable solvent in the presence of a base at a temperature from 20° C. to the reflux temperature of the solvent to give the compound of formula (VIIa)

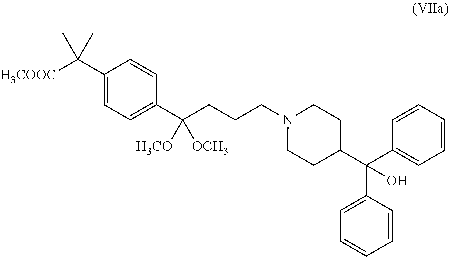

c′) reacting the compound (VIIa) with an aqueous acid in the presence of a suitable solvent at a temperature from 20° C. to 40° C. to give the compound of formula (VIIIa)

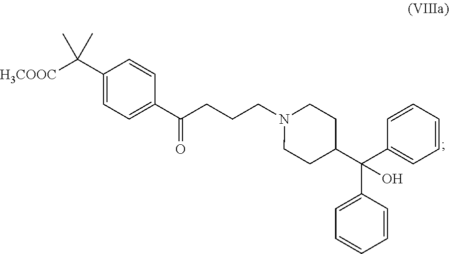

and
d′) transforming the compound (VIIIa) into fexofenadine.

The transformation of compounds (VIIIa) into fexofenadine is carried out according to the method described in WO 2011/158262 in the name of the Applicant.

The trialkylorthoformate in step a′) is selected from among trimethylorthoformate, triethylorthoformate or triisopropylorthoformate. Trimethylorthoformate is preferably used.

The alcoholic solvent is a linear or branched C₁-C₄ alcohol, preferably selected from among methanol, ethanol, isopropanol, butanol. Methanol is preferably used.

The acid catalyst in step a′) is preferably selected from sulfuric acid, camphorsulfonic acid and methansulfonic acid. Camphorsulfonic acid is preferably used.

The advent in step b′) is preferably selected from among toluene, acetonitrile, tetrahydrofuran. Toluene is preferably used.

The base is preferably selected from among sodium bicarbonate, potassium bicarbonate, triethylamine. Sodium bicarbonate is preferably used.

The aqueous acid in step c′) is preferably selected from among sulfuric acid, hydrochloric acid. Sulfuric acid is preferably used.

The solvent in step c′) is preferably selected from among methanol, ethanol, isopropanol. Methanol is preferably used.

A still more preferred embodiment of the present invention is a process for the preparation of fexofenadine comprising:
1) transforming an intermediate of formula (VIa) into a compound of formula (Vd)

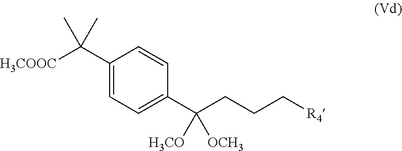

wherein
R₄′ is a halogen atom: by reaction with a trialkylorthoformate in an alcoholic solvent in the presence of an acid catalyst at a temperature from 25° C. to the reflux temperature of the solvent;
2) reacting the resultant compound of formula (Vd) with azacyclonol in a suitable solvent in the presence of a base at a temperature from 20° C. to the reflux temperature of the solvent, followed by the optional saponification and by the optional subsequent treatment with acetic acid to give a compound of formula (VIIa)

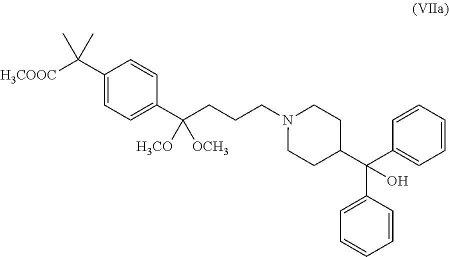

3) saponifying the compound (VIIa) with a base in an alcoholic solvent and subsequently treating with acetic acid to give the compound of formula (VIIc)

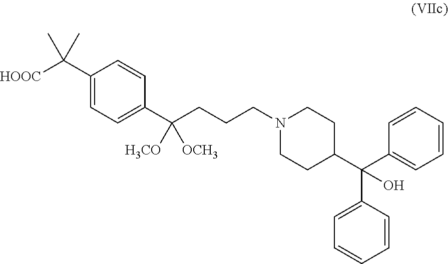 [SMILES: COC(CCCN1CCC(C(O)(c2ccccc2)c2ccccc2)CC1)(OC)c1ccc(C(C)(C)C(=O)O)cc1]

4) deacetalizing the compound of formula by treatment with a strong aqueous acid in an alcoholic solvent at a temperature from 20° C. to 40° C.; and
5) transforming the resultant compound into fexofenadine.

The trialkylorthoformate in step 1) is selected from among trimethylorthoformate, triethylorthoformate or triisopropylorthoformate. Trimethylorthoformate is preferably used.

The alcoholic solvent is a linear or branched C₁-C₄ alcohol, preferably selected from among methanol, ethanol, isopropanol, butanol. Methanol is preferably used.

The acid catalyst in step 1) is preferably selected from sulfuric acid, camphorsulfonic acid and methansulfonic acid. Camphorsulfonic acid is preferably used.

The solvent in step 2) is preferably selected from among toluene, acetonitrile, tetrahydrofuran. Toluene is preferably used.

The base is preferably selected from among sodium bicarbonate, potassium bicarbonate, triethylarnine. Sodium bicarbonate is preferably used.

The saponification reaction is preferably carried out in the presence of sodium or potassium hydroxide in a solvent selected from among methanol, ethanol, isopropanol. Methanol is preferred.

The aqueous acid in step 4) is preferably selected from among sulfuric acid, hydrochloric acid. Hydrochloric acid is preferably used.

The solvent in step 4) is preferably selected from among methanol, ethanol, isopropanol. Methanol is preferably used.

The compounds (Vd), (VIIa) and (VIIc)

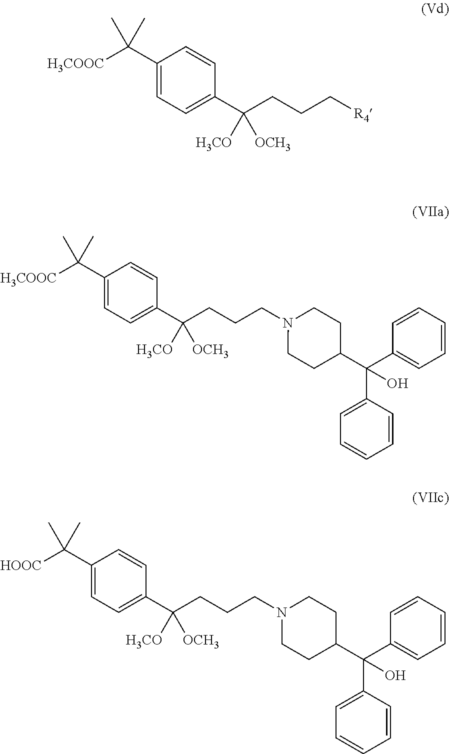

wherein R₄′ is a halogen atom, preferably chlorine or bromine, are new and a preferred object of the present invention.

All the terms used in the present description, unless otherwise indicated, are intended in their common meaning as known in the art. Other more specific definitions for some terms, as used in the present context, are underlined herein after and constantly apply in the whole description and claims, unless a different definition explicitly provides for a broader definition.

The term “alkyl” refers to a linear or branched hydrocarbon, containing from 1 to 20 carbon atoms. Examples of alkyl groups include, but are not limited to, methyl, ethyl, n-propyl, isopropyl, n-butyl, sec-butyl, isobutyl, tert-butyl, nm pentyl, n-hexyl, etc. A preferred alkyl group of the present invention is n-butyl.

Although the present invention has been described in its characterizing features, changes and equivalents which are obvious to the skilled in the art are included in the present invention.

### DETAILED DESCRIPTION OF THE INVENTION

Herein after, the present invention will be illustrated by some examples which are not intended to limit the scope of the invention.

### EXAMPLE 1

### Synthesis of methyl 2-(4-(2-methoxytetrahydrofuran-2-yl)phenyl)-2-methyl-propanoate (Compound Vb)

In a reaction flask 10 g of methyl 2-(4-(4-hydroxybut-1-inyl)-2-methyl-propanoate (0.04 mol), 30 ml methanol, 16.6 g zinc chloride (0.12 mol) were charged and the reaction mixture was kept under stirring up to complete dissolution. Then, 27 ml hydrochloric acid 3M in methanol (0.08 mol) were added and the reaction mixture was heated to the reflux temperature and kept under these conditions for six hours. At the end of the reaction, the temperature was brought to 15° C., a 30% ammonia solution was added up to pH 8 and the resultant solid was filtered. To the mother liquor 100 ml toluene were added, the mixture was washed with water (2×20 ml) and the collected organic phases were concentrated to residue by distillation under vacuum to give 10 g of methyl 2-(4-(2-methoxytetrahydrofuran-2-yl)phenyl)-2-methyl-propanoate.

¹H-NMR (CDCl₃, 300 MHz): δ 7.90 (d, 2H), 7.36 (d, 2H), 3.61 (s, 3H), 3.41 (t, 2H), 3.28 (s, 3H), 3.00 (t, 2H), 1.96 (m, 2H), 1.55 (s, 6H).

¹³C-NMR (CDCl₃, 300 MHz): δ 195.50 (C), 176.64 (C), 149.88 (C), 135.55 (C), 128.66 (CH), 125.99 (CH), 71.79 (CH₂), 58.54 (CH₃), 52.37 (CH₃), 46.87 (C), 35.09 (CH₂), 26.44 (CH₃), 24.20 (CH₂).

MS (m/e): 246.1 (M-CH₃OH), 220.1, 205.1, 161.1 (100%), 146.1, 131.1, 118.1.

### EXAMPLE 2

### Synthesis of methyl 2-(4-(2-methoxytetrahydrofuran-2-yl)phenyl)-2-methyl-propanoate (Compound Vb)

In a reaction flask 1.0 g of methyl 2-(4-(4-hydroxybut-1-inyl)-2-methyl-propanoate (0.004 mol), 5 ml methanol, 1.68 g zinc chloride (0.01 mol) were charged and the reaction mixture was kept under stirring up to complete dissolution. Then, 0.80 g hydrochloric acid 37% in water (0.008 mol) were added and the reaction mixture was heated to the reflux temperature and kept under these conditions for eight hours. At the end of the reaction, the temperature was brought to 15° C., a 30% ammonia solution was added up to pH 8 and the resultant solid was filtered. To the mother liquor 10 ml toluene were added, the mixture was washed with water (2×5 ml) and the collected organic phases were concentrated to residue by distillation under vacuum to give 1 g of methyl 2-(4-(2-methoxytetrahydrofuran-2-yl)phenyl)-2-methyl-propanoate.

### EXAMPLE 3

### Synthesis of methyl 2-(4-(2-methoxytetrahydrofuran-2-yl)phenyl)-2-methyl-propanoate (Compound Vb)

In a reaction flask 1.0 g of methyl 2-(4-(4-hydroxybut-1-inyl)-2-methyl-propanoate (0.004 mol), 5 ml methanol, 2.73 g zinc bromide (0.01 mol) were charged and the reaction mixture was kept under stirring up to complete dissolution. Then, 2.0 g hydrobromic acid 33% solution in acetic acid (0.008 mol) were added and the reaction mixture was heated to the reflux temperature and kept under these conditions for eight hours. At the end of the reaction, the temperature was brought to 15° C., a 30% ammonia solution was added up to pH 8 and the resultant solid was filtered. To the mother liquor 10 ml toluene were added, the mixture was washed with water (2×5 ml) and the collected organic phases were concentrated to residue by distillation under vacuum to give 1.1 g of methyl 2-(4-(2-methoxytetrahydrofuran-2-yl)phenyl)-2-methyl-propanoate.

### EXAMPLE 4

### Synthesis of methyl 2-(4-(2-methoxytetrahydrofuran-2-yl)phenyl)-2-methyl-propanoate (Compound Vb)

In a reaction flask 10 g of methyl 2-(4-(4-hydroxybut-1-inyl)-2-methyl-propanoate (0.04 mol), 50 ml methanol, 27.4 g zinc bromide (0.12 mol) were charged and the reaction mixture was kept under stirring up to complete dissolution. Then, 13.5 g hydrobromic acid 48% solution in water (0.08 mol) were added and the reaction mixture was heated to the reflux temperature and kept under these conditions for eight hours. At the end of the reaction, the temperature was brought to 15° C., a 30% ammonia solution was added up to pH 8 and the resultant solid was filtered. To the mother liquor 100 ml toluene were added, the mixture was washed with water (2×20 ml) and the collected organic phases were concentrated to residue by distillation under vacuum to give 11 g of methyl 2-(4-(2-methoxytetrahydrofuran-2-yl)phenyl)-2-methyl-propanoate.

### EXAMPLE 5

### Synthesis of methyl 2-(4-(2-methoxytetrahydrofuran-2-yl)phenyl)-2-methyl-propanoate (Compound Vb)

In a reaction flask 0.8 g zinc (0.01 mol), 5 ml methanol, 7.46 g hydrobromic acid 33% solution in acetic acid (0.03 mol) were charged and the temperature was brought to 45° C. up to complete dissolution. Then. 1.0 g of methyl 2-(4-(4-hydroxybut-1-inyl)-2-methyl-propanoate (0.004 mol) were added and the temperature was brought to the reflux temperature of the solvent and kept under these conditions for eight hours. At the end of the reaction, the temperature was brought to 15° C., a 30% ammonia solution was added up to pH 8 and the resultant solid was filtered. To the mother liquor 10 ml toluene were added, the mixture was washed with water (2×5 ml) and the collected organic phases were concentrated to residue by distillation under vacuum to give 1.1 g of methyl 2-(4-(2-methoxytetrahydrofuran-2-yl)phenyl)-2-methyl-propanoate.

### EXAMPLE 6

### Synthesis of methyl 4-(4-bromo-1-oxybutyl)-α,α-dimethylphenylacetate (Compound VI)

In a reaction flask 10 g of methyl 2-(4-(2-methoxytetrahydrofuran-2-yl)phenyl)-2methyl-propanoate (0.038 mol) and 20 ml toluene were charged. To the resultant solution 17.6 g hydrobromic acid 33% soiution in acetic acid (0.072 mol) were added dropwise and the reaction mixture was kept under stirring at the temperature of 25° C. for 10 hours. At the end of the reaction, 30 ml toluene were added, the mixture was washed with a saturated sodium bicarbonate solution (1×30 ml) and water (2×20 ml). The collected organic phases were concentrated to residue by distillation under vacuum to give 10.5 g di methyl 4-(4-bromo-1-oxybutyl)-α,α-dimethylphenylacetate.

¹H-NMR (CDCl₃, 300 MHz): δ 7.96 (d, 2H), 7.42 (d, 2H), 3.58 (s, 3H), 3.54 (t, 2H), 3.17 (t, 2H), 2.99 (m, 2H), 1.58 (s, 6H).

### EXAMPLE 7

### Synthesis of methyl 4-(4-bromo-1-oxybutyl)-α,α-dimethylphenylacetate (Compound VI)

In a reaction flask 1.0 g of methyl 2-(4-(4-hydroxybut-1-inyl)-2-methyl-propanoate (0.004 mol), 2 ml toluene, 1.36 g hydrobromic acid 48% solution in water (0.008 mol) were charged and the reaction mixture was kept under stirring at the temperature of 25° C. for 15 hours. At the end of the reaction, 3 ml toluene were added, the mixture was washed with a saturated sodium bicarbonate solution (1×3 ml) and water (2×2 ml). The collected organic phases were concentrated to residue by distillation under vacuum and purified by column chromatography to give 0.45 g di methyl 4-(4-bromo-1-oxybutyl)-α,α-dimethylphenylacetate.

### EXAMPLE 8

### Preparation of methyl 2-(4-(4-(4-(hydroxydiphenylmethyl)piperidin-1-yl)-1-oxybutyl)phenyl)-2-methyl-propanoate

In a reaction flask 5 g methyl 4-(4-bromo-1-oxybutyl)-α,α-dimethylphenylacetate (0.15 mol), 25 ml toluene, 4.08 g azacyclonol (0.015 mol), 10 ml water, 1.53 g sodium bicarbonate (0018 mol) and 0.25 g potassium iodide (0.0015 mol) were charged, the reaction mixture was brought to the reflux temperature and kept under these conditions for 20 hours. At the end of the reaction, the temperature was brought to 25° C., the separated organic phase was concentrated to residue by distillation under vacuum to give 7 g methyl 2-(4-(4-(4-(hydroxy-diphenylmethyl)piperidin-1-yl)-1-oxybutyl)phenyl)-2-methyl-propanoate.

### EXAMPLE 9

### Synthesis of fexofenadine

In a reaction flask, 7.0 g methyl 2-(4-(4-(4-(hydroxydiphenylmethyl)piperidin-1-yl)-1-oxybutyl)phenyl)-2-methyl-propanoate (0.01 mol), 20 ml methanol, 2.0 g sodium hydroxide 30% solution in water (0.015 mol) were charged, the temperature were brought to the reflux temperature of the solvent and the reaction mixture was kept under these conditions for seven hours. At the end of the reaction, the temperature was brought to 10° C., 0.2 g sodioborohydride (0.005 mol) were charged and the temperature was brought to 40° C. and kept under these conditions for fifteen hours.

At the end of the reaction, the temperature was brought to 15° C. and 1.2 g acetic acid (0.02 mol) and 10 ml water were charged. The suspension was filtered and the solid was washed with a 1:1 water/methanol mixture (2×4 ml). The solid was dried under vacuum at 40° C. obtaining 6.7 g fexofenadine.

### EXAMPLE 10

### Preparation of methyl 2-(4-(4-bromo-1,1-dimethoxybutyl)phenyl)-2-methyl-propanoate (Compound Vd)

In a reaction flask, 7 g methyl 4-(4-bromo-1-oxybutyl)-α,α-dimethylphenylacetate (0.02 mol), 35 ml methanol, 0.2 g sulfuric acid (0.002 mol) and 3.4 g trimethylorthoformate (0.032 mol) were charged, the solution was heated to the reflux temperature and kept under these conditions for ten hours. At the end of the reaction, the temperature was brought to 25° C. and 6.1 g sodium methoxide 30% solution hi methanol (0.034 mol), 40 ml methylene chloride were added and the organic phase was washed with water (2×10 ml). The collected organic phases were concentrated to residue by distillation under vacuum to give 7.2 g methyl 2-(4-(4-bromo-1 1-dimethoxybutyl)phenyl)-2-methyl-propanoate.

¹H-NMR (CDCl₃, 300 MHz): δ 7.37 (d, 2H), 7.29 (d, 2H), 3.63 (s, 3H), 3.18 (m, 4H), 2.02 (m, 2H), 1.55 (s, 6H).

¹³C-NMR (CDCl₃, 300 MHz): δ 177.25 (C), 144.24 (C), 138.94 (C), 126.13 (CH), 125.39 (CH), 103.02 (C), 52.25 (CH₃), 48.73 (CH₃), 46.41 (C), 35.89 (CH₂), 33.74 (CH₂), 27.13 (CH₂), 26.64 (CH₃).

### EXAMPLE 11

### Synthesis of methyl 2-(4-(4(4-(hydroxydiphenylmethyl)piperidin-1-yl)-1,1-dimethoxybutyl)phenyl)-2-methyl-propanoate (Compound VIIa)

In a reaction flask 5 g methyl 2-(4-(4-bromo-1,1-dimethoxybutyl)phenyl)-2-methyl-propanoate (0.013 mol), 25 ml toluene, 3.57 g azacyclonol (0.013 mol), 10 ml water, 1.30 g sodium bicarbonate (0.016 mol) and 0.21 g potassium iodide (0.0013 mol) were charged, the reaction mixture was heated to the reflux temperature and kept under these conditions for 20 hours. At the end of the reaction, the temperature was brought to 25° C., the separated organic phase was concentrated to residue by distillation under vacuum to give 7.0 g methyl 2-(4-(4-(4-(hydroxy-diphenylmethyl)piperidin-1-yl)-1,1-dimethoxybutyl)phenyl)-2-methyl-propanoate.

¹H-NMR (CDCl₃, 300 MHz): δ 7.37 (m, 4H), 7.29 (m, 8H), 3.63 (s, 3H), 3.15 (s, 6H), 3.01 (m, 2H), 2.71 (m, 5H), 1.88 (m, 2H), 1.55 (s, 6H), 1.36 (m, 2H).

### EXAMPLE 12

### Synthesis of methyl 2-(4-(4-(4-(hydroxydiphenylmethyl)piperidin-1-yl)butanoyl)phenyl)-2-methyl-propanoate (Compound VIII)

In a reaction flask 7.0 g methyl 2-(4-(4-(4-(hydroxydiphenylmethyl)piperidin-1-yl)-1,1-dimethoxybutyl)phenyl)-2-methylpropanoate (0.01 mol), 35 ml methanol, 5 ml water, 1.5 g sulfuric acid 96% (0.015 mol) were charged, the temperature was brought to 40° C. and the reaction mixture was kept under these conditions for twelve hours. At the end of the reaction, the temperature was brought to 15° C., 20 ml water and ammonia 30% solution up to pH 8 were added, the resultant solid was filtered and dried in oven at 40° C. under vacuum to give 5.8 g methyl 2-(4-(4-(4-(hydroxydiphenylmethyl)piperidin-1-yl)butanoyl)phenyl)-2-methyl-propanoate.

### EXAMPLE 13

### Synthesis of fexofenadine

The synthesis of fexofenadine starting from methyl 2-(4-(4-(4-(hydroxydiphenylmethyl)piperidin-1-yl)butanoyl)phenyl)-2-methyl-propanoate, as prepared in example 12, was carried out following the procedure reported in example 9.

### EXAMPLE 14

### Preparation of methyl 4-(4-bromo-1-oxybutyl)-α,α-dimethylphenylacetate (Compound VI)

In a reaction flask 20 g hydrobromic acid 33% in acetic acid (0.082 mol) and 10 g methyl 2-(4-(4-hydroxybut-1-inyl)-2-methyl-propanoate (0.04 mol) were charged keeping the temperature at 35° C. and the reaction mixture was kept under these conditions for four hours. At the end of the reaction, the temperature was brought to 20° C., 50 ml toluene were added and the organic phase was washed with water (3×25 ml). The collected organic phases were concentrated to residue by distillation under vacuum to give 10.6 g methyl 4-(4-bromo-1-oxybutyl)-α,α-dimethylphenylacetate.

¹H-NMR (CDCl₃, 300 MHz): δ 7.96 (d, 2H), 7.42 (d, 2H), 3.58 (s, 3H), 3.54 (t, 2H), 3.17 (t, 2H), 2.99 (m, 2H), 1.58 (s, 8H).

### EXAMPLE 15

### Preparation of 2-(4-(4-(4-(hydroxydiphenylmethyl)piperidin-1-yl)-1,1-dimethoxybutyl)phenyl-2-methyl-propanoic acid (Compound VIIc)

In a reaction flask 102 g methyl 2-(4-(4-(4-(hydroxydiphenylmethyl)piperidin-1-yl)-1,1-dimethoxybutyl)phenyl)-2-methylpropanoate (0.18 mol), 270 ml methanol and 40.8 g sodium hydroxide 30% solution (0.31 mol) were charged, the reaction mixture was heated to the reflux temperature of the solvent and kept under these conditions for four hours. At the end of the reaction, the temperature was brought to 20° C. and 18.4 g glacial acetic acid (0.31 mol) were added. The precipitated solid was filtered and washed with methanol (2×30 ml) and dried in oven under vacuum at 40° C. up to constant weight to give 80 g 2-(4-(4-(4-(hydroxydiphenylmethyl)piperidin-1-yl)-1,1-dimethoxybutyl)phenyl)-2-methyl-propanoic acid.

¹H-NMR (DMSO, 300 MHz): δ 7.48 (d, 4H), 7.31 (s, 4H), 7.23 (m, 4H), 7.10 (m, 2H), 3.02 (s, 6H), 2.60 (d, 2H), 2.40 (m, 1H), 2.03 (m, 2H), 1.77 (m, 4H), 1.42 (s, 6H), 1.36 (m, 2H), 1.68 (m, 2H), 0.99 (m, 2H).

### EXAMPLE 16

### Preparation of fexofenadine

In a reaction flask, 10 g 2-(4-(4-(4-(hydroxydiphenylmethyl)piperidin-1-yl)-1,1-dimethoxybutyl)phenyl)-2-methyl-propanoic acid (0.018 mol), 30 ml methanol and 2 g hydrochloric acid 37% solution (0.018 mol) were charged, the reaction mixture was heated to 25° C. and kept under these conditions for one hour. At the end of the reaction, 5.0 g sodium hydroxide 30% solution in water (0.038 ml) and 0.4 g sodioborohydride (0.010 mol) were charged, the temperature was brought to 40° C. and the reaction mixture was kept under these conditions for ten hours. At the end of the reaction, the temperature was brought to 13° C. and 1.2 g acetic acid (0.02 mol) and 10 ml water were charged. The is suspension was filtered and the solid was washed with a 1:1 water/methanol mixture (2×4 ml). The solid was dried under vacuum at 40° C. obtaining 9 g fexofenadine.

### EXAMPLE 17

### Synthesis of methyl 4-(4-chloro-1-oxybutyl)-α,α-dimethylphenylacetate

In a reaction flask, 50 g methyl 2-(4-(4-hydroxybut-1-inyl)-2-methyl-propanoate (0.203 mol), 50 ml toluene were charged, the temperature was brought to 40° C. and 50.01 g hydrochloric acid 37% solution in water (0.507 mol) were charged in eight hours and the reaction mixture was kept under these conditions for about four hours. At the end of the reaction, the temperature was brought to 25° C., 100 ml toluene were added and the mixture was washed with water (4×50 ml) up to a pH value of about 4. The collected organic phases were concentrated to residue by distillation under vacuum to give 54 g methyl 4-(4-chloro-1-oxybutyl)-α,α-dimethylphenylacetate.

### EXAMPLE 18

### Synthesis of methyl 2-(4-(4-chloro-1,1-dimethoxybutyl)phenyl)-2-methyl-propanoate

In a reaction flask, 25 g methyl 4-(4-chloro-1-oxybutyl)-α,α-dimethylphenylacetate (0.088 mol), 50 ml methanol, 11.27 g trimethylorthoformate (0.106 mol), 0865 g sulfuric acid (0.009 mol) were charged, the solution was heated to the reflux temperature and kept under these conditions for three hours. At the end of the reaction, the temperature was brought to 25° C. and 1.58 g sodium methoxide 30% solution in methanol were added, the solvent was removed by distillation under vacuum and 100 ml toluene were added. After washing with water (1×50 nil) the collected organic phases were concentrated to residue by distillation under vacuum to give 34 g methyl 2-(4-(4-chloro-1,1-dimethoxybutyl)phenyl)-2-methyl-propanoate.

¹H-NMR (CDCl₃, 300 MHz): δ 7.40 (m, 2H), 7.37 (m, 2H), 3.64 (s, 3H), 3.37 (t, 2H), 3.14 (s, 6H), 2.01 (m, 2H), 1.57 (s, 6H), 1.45 (m, 2H).

### EXAMPLE 19

### Synthesis of methyl 2-(4-(4-(4-(hydroxydiphenylmethyl)piperidin-1-yl)-1,1-dimethoxybutyl)phenyl)-2-methyl-propanoate

In a reaction flask 34 g methyl 2-(4-(4-chloro-1,1-dimethoxybutyl)phenyl)-2-methyl-propanoate (0.079 mol), 130 ml toluene, 21.17 g azacyclonol (0.079 mol), 10 ml water, 7.39 g sodium bicarbonate (0.088 mol) and 0.050 g potassium iodide (0.003 mol) were charged, the reaction mixture was heated to the reflux temperature and kept under these conditions for 15 hours. At the end of the reaction, the temperature was brought to 25° C., the separated organic phase was concentrated to residue by distillation under vacuum to give 50 g methyl 2-(4-(4-(4-(hydroxy-diphenylmethyl)piperidin-1-yl)-1,1-dimethoxybutyl)phenyl)-2-methyl-propanoate.

¹H-NMR (CDCl₃, 300 MHz): δ 7.37 (m, 4H), 7.29 (m, 8H), 3.63 (s, 3H), 3.15 (s, 6H), 3.01 (m, 2H), 2.71 (m, 5H), 1.88 (m, 2H), 1.55 (s, 6H), 1.36 (m, 2H).

1. A compound of formula (V) 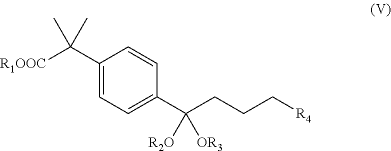
wherein
R₁, R₂ and R₃, the same or different, are linear or branched C₁-C₂₀ alkyl groups; or
R₂ and R₃ linked together are a five or six membered ring of formula: 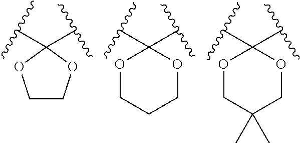
R₄ is a halogen atom or a hydroxy group; or
the groups OR₂ and R₄ linked together are a five membered cycle of formula:

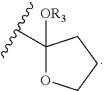

2. A compound according to claim 1 of formula (Va) 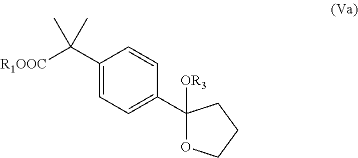
wherein
R₁ and is R₃, the same or different, are linear or branched C₁-C₂₀ alkyl groups; 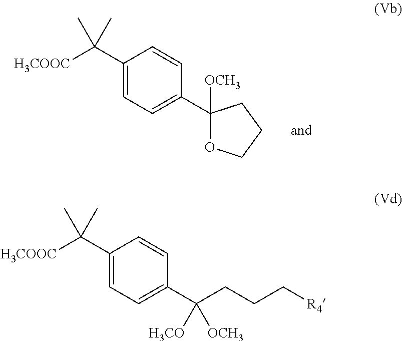
wherein
R₄′ is a halogen atom,

3. A process for the synthesis of a compound of claim 1, comprising dissolving intermediates of formula (IIa) 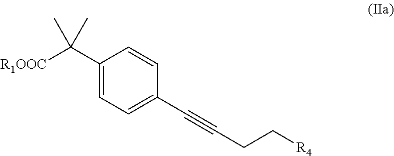
wherein
R₁ is a linear or branched C₁-C₂₀ alkyl group: and
R₄ is a halogen or a hydroxy group, in an alcoholic solvent and reacting the resulting solution with a Brønsted acid, or a Lewis acid, or mixtures thereof, at a temperature from 25° C. to the reflux temperature of the solvent.

4. A process according to claim 3, wherein the solvent is a linear or branched C₁-C₄ alcohol.

5. A process according to claim 3, wherein the Brønsted acid is selected from the group consisting of hydrochloric acid, hydrobromic acid and sulfuric acid.

6. A process according to claim 3, wherein the Lewis acid is a zinc salt.

7. A process according to claim 3, wherein a mixture of a Brønsted acid and a Lewis acid is used.

8. A process according to claim 7, wherein a mixture of hydrochloric acid and zinc chloride is used.

9. A process for the preparation of the compounds of formula (VI), starting from a compound of formula (IIa) according to the following scheme: 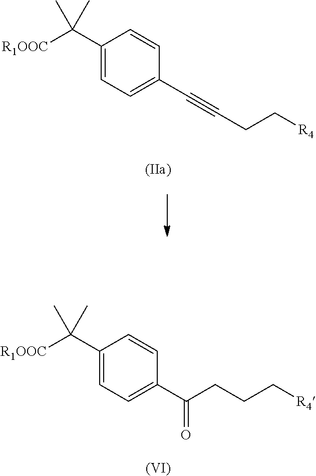
wherein
R₁ is a linear or branched C₁-C₂₀ alkyl group:
R₄ is a halogen atom or a hydroxy group; and
R₄′ is a halogen atom;
by reaction with a hydrogen halide solution in a suitable solvent at a temperature from 30° C. to 50° C.

10. A process according to claim 9, wherein the hydrogen halide solution is a solution of hydrobromic acid in acetic acid or an aqueous hydrochloric acid solution.

11. A process according to claim 9, for the preparation of compounds of formula (VIa) 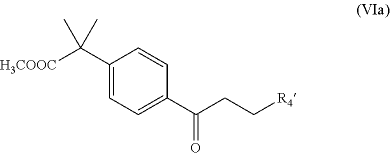
wherein
R₄′ is chlorine or bromine,

12. A process for the synthesis of fexofenadine, starting from intermediates of formula (V), comprising:
reacting an intermediate of formula (V) with aqueous hydrobromic acid or acetic acid in the presence of a solvent to give an intermediate of formula (VI) 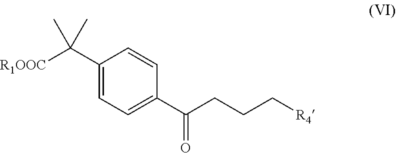
wherein
R₁ is a linear or branched C₁-C₂₀ alkyl group and R₄′ is a halogen atom; and
transforming the intermediate of formula (VI) into fexofenadine.

13. A process according to claim 12 starting from intermediates of formula (Va).

14. A process for the synthesis of fexofenadine, starting from intermediates of formula (VI), comprising:
a) transforming an intermediate of formula (VI) into a compound of formula (Vc) 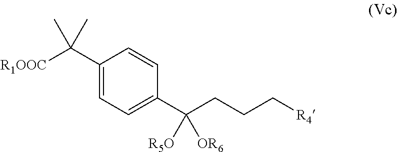
wherein
R₁, R₅ and R₆, the same or different, are linear or branched C₁-C₂₀ alkyl groups; by reaction with a trialkylorthoformate in an alcoholic solvent in the presence of an acid catalyst at a temperature from 25° C. to the reflux temperature of the solvent;
b) reacting the resultant compound of formula (Vc) with azacyclonol in a suitable solvent in the presence of a base at a temperature from 20° C. to the reflux temperature of the solvent, followed by the optional saponification and by the optional subsequent treatment with acetic acid to give a compound of formula (VII) 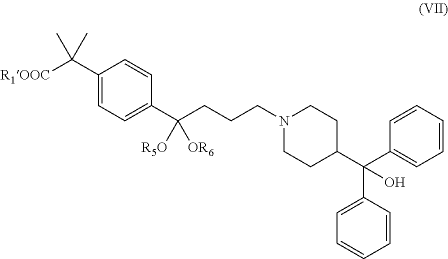
wherein
R₁ is a hydrogen atom or a linear or branched C₁-C₂₀ alkyl group; and
R₅ and R₆, the same or different, are linear or branched C₁-C₂₀ alkyl groups;
c) reacting the resultant compound (VII) with an aqueous acid in the presence of a suitable solvent at a temperature from 20° C. to 40° C. to give the compound of formula (VIII) 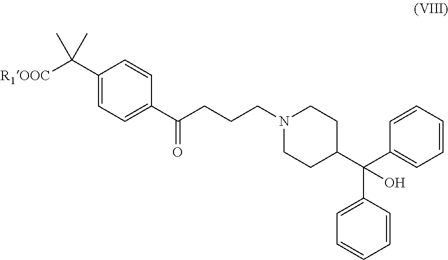
wherein
R₁′ is a hydrogen atom or a linear or branched C₁-C₂₀ alkyl group; and
d) transforming the resultant compound (VIII) into fexofenadine.

15. A process according to claim 14, wherein the trialkylorthoformate is selected from the group consisting of trimethylorthoformate, triethylorthoformate and triisopropylorthoformate.

16. A process according to claim 14, wherein the alcoholic solvent is a linear or branched C₁-C₄ alcohol.

17. A process according to claim 14, wherein the acid catalyst is selected from the group consisting of sulfuric acid, camphorsulfonic acid and methansulfonic acid.

18. A process according to claim 14, wherein the solvent in step b) is selected from the group consisting of toluene, acetonitrile and tetrahydrofuran.

19. A process according to claim 14, wherein the base is selected from the group consisting of sodium bicarbonate, potassium bicarbonate and triethylamine.

20. A process according to claim 14, wherein the aqueous acid in step c) is sulfuric acid or hydrochloric acid.

21. A process according to claim 14, starting from intermediates of formula (VIa), 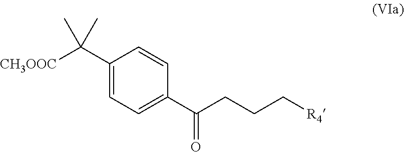
wherein
R₄′ is a halogen atom;
comprising:
a′) transforming an intermediate of formula (VIa) into a compound of formula (Vd) 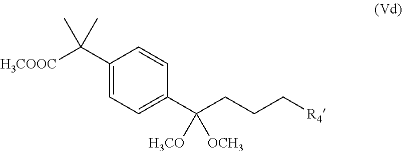
wherein
R₄′ is a halogen atom;
by reaction with a trialkylorthoformate in an alcoholic solvent in the presence of an acid catalyst at a temperature from 25° C. to the reflux temperature of the solvent;
b′) reacting the resultant compound of formula (Vd) with azacyclonol in a suitable solvent in the presence of a base at a temperature from 20° C. to the reflux temperature of the solvent to give the compound of formula (VIIa) 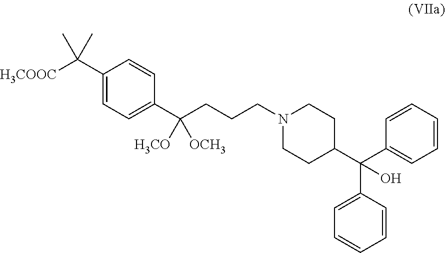
c′) reacting the compound (VIIa) with an aqueous acid in the presence of a suitable solvent at a temperature from 20° C. to 40° C. to give the compound of formula (VIIIa) 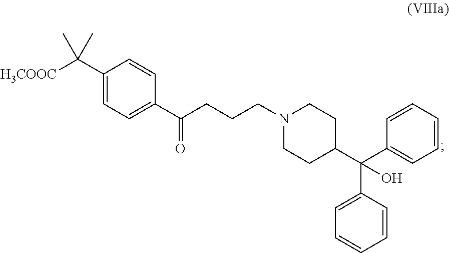
and
d′) transforming the compound (VIIIa) into fexofenadine.

22. A process according to claim 14, comprising:
a) transforming an intermediate of formula (VIa) into a compound of formula (Vd) 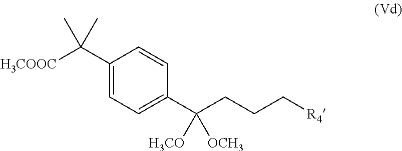
wherein
R₄′ is a halogen atom; by reaction with a trialkylorthoformate in an alcoholic solvent in the presence of an acid catalyst at a temperature from 25° C. to the reflux temperature of the solvent;
b) reacting the resultant compound of formula (Vd) with azacyclonol in a suitable solvent in the presence of a base at a temperature from 20° C. to the reflux temperature of the solvent, followed by the optional saponification and by the optional subsequent treatment with acetic acid to give a compound of formula (VII) 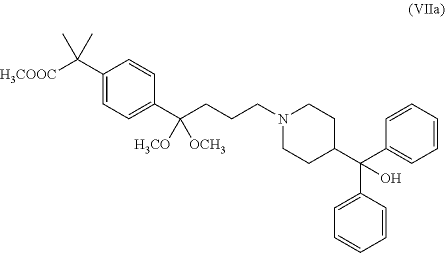
c) saponifying the compound (VIIa) with a base in an alcoholic solvent and subsequently treating with acetic acid to give the compound of formula (VIIc) 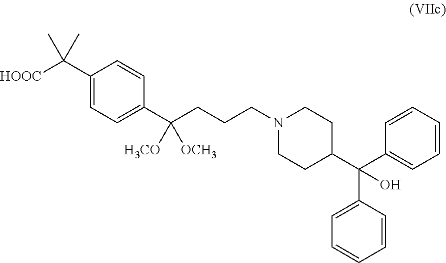 [SMILES: COC(CCCN1CCC(C(O)(c2ccccc2)c2ccccc2)CC1)(OC)c1ccc(C(C)(C)C(=O)O)cc1]
d) deacetalizing the compound of formula (VIIc) by treatment with a strong aqueous acid in an alcoholic solvent at a temperature from 20° C. to 40° C.
wherein
R₁ is a hydrogen atom or a linear or branched C₁-C₂₀ alkyl group; and
e) transforming the resultant compound into fexofenadine.

23. A compound of formula (VIIa).

24. A compound of formula (VIIc).
